(19) United States
(12) Patent Application Publication
(10) Pub. No.: US 2005/0054940 A1
(43) Pub. Date: Mar. 10, 2005

(54) Apparatus and method for monitoring heart rate variability

(76) Inventor: Adam J. Almen, Minneapolis, MN (US)

Correspondence Address: ALTERA LAW GROUP, LLC, 6500 CITY WEST PARKWAY, SUITE 100, MINNEAPOLIS, MN, 55344-7704, US

(21) Appl. No.: 10/829,544

(22) Filed: Apr. 22, 2004

Related U.S. Application Data
(60) Provisional application No. 60/464,762, filed on Apr. 23, 2003.

(51) Int. Cl. A61B 5/0402

(52) U.S. Cl. 600/509

(57) ABSTRACT

A wrist-worn heart rate variability monitor is provided. Heart rate variability (“HRV”) refers to the interval between heart beats and is a reflection of an individual's current health status. Over time, an individual may use the results of HRV tests to monitor either improvement or deterioration of specific health issues. Thus, one use of the HRV test is as a medical motivator. When an individual has a poor HRV result, it is an indicator that they should consult their physician and make appropriate changes where applicable to improve their health. The inventive monitor is capable of monitoring the stages of sleep by changes in the heart rate variability and can record the sleep (or rest) sessions with the resulting data accessible by the user or other interested parties. The inventive monitor is thus capable of several novel uses: (1) to assist the user with a nap that allows predetermined time in one or more sleep stages; (2) determination of the duration of a sleep session, including length of time spent in one or more sleep stages; (3) in concert with a home's central electronic and computer control unit, the device uses HRV to determine when the house may be placed in “sleep” mode and when it is appropriate to place the house in “awake mode”; and (4) performance of an HRV test.

### RELATED APPLICATIONS

This application claims priority to provisional application No. 60/464,762, entitled Wrist Heart Rate Variable Monitor filed Apr. 23, 2003.

### FIELD OF THE INVENTION

This invention relates generally to monitoring heart rate variability using a wrist worn monitor.

### BACKGROUND OF THE PRESENT INVENTION

This invention monitors a user's heart rate variability (HRV). The invention also performs a heart rate variability test. Heart rate variability refers to the interval between heart beats and may be mathematically defined as the one sigma standard deviation of the heart rate about the mean heart rate value. A heart rate variability test is a reflection of a person's current health status. By taking heart rate variability tests over time, an individual is able to gauge improvement or deterioration in their health status. Such improvements or deterioration of health may result from a number of sources including, e.g., changes in lifestyle such as smoking cessation, starting an exercise program, surgery recovery, stressor additions or removals, diet changes. Thus, in this context, the HRV test may be used as a medical motivator. The HRV test may also be used as an early indicator diagnostic tool. For example, the HRV test has been demonstrated to have prognostic associations with future coronary disease.

Human sleep is described as a succession of recurring stages, including an awake stage, non-REM stages and the REM stage. The awake stage in this context is actually the phase during which a person begins the process of falling asleep. Sleep quality changes with the transition from one sleep stage into another. Significantly for purposes of this invention, the transition from stage to stage is marked with observable, though subtle, changes in bodily function, including heart rate variability.

Analysis of 24-hour HRV typically shows a nocturnal increase in the standard deviation of heart beat intervals. The heart rate is further known to decrease relatively rapidly as a person transitions from the awake stage to the non-REM stages. As the individual eventually transitions from the non-REM sleep stages to REM sleep, the heart rate becomes more erratic and the variability increases. There are several stages of REM sleep, each marked by changes in heart rate variability. The first REM stage typically lasts about 10 minutes, with each recurring REM stage lengthening, with the final stage lasting about one hour. The inventive monitor is capable of detecting the heart rate variability within each sleep stage as well as the transition from one sleep stage to the next, i.e., the transition from awake to non-REM sleep, the transition from non-REM sleep to REM sleep, and the completion of an REM sleep stage and subsequent transition to the next REM sleep stage.

Finally, sleep apnea is a condition whereby afflicted individuals literally stop breathing repeatedly during sleep, often for a minute or longer and as many as hundreds of times during a single night's sleep. Very often individuals with sleep apnea experience disrupted sleep and are prevented from reaching the later stages of sleep, such as REM sleep, which the body requires for rest and replenishment of strength. Heart rate variability data can be used to assist the physician in diagnosing and monitoring the efficacy of treatment regimens for sleep apnea. The inventive monitor may be used to determine whether heart rate variability indicates that sleep is continually interrupted and whether a sufficient amount of REM sleep is being obtained.

A wrist worn heart rate variability monitor for use in the above-mentioned conditions is desirable. The inventive monitor is used in four basic applications. The first application is used to assist the user with a timed nap. The heart rate variability data obtained through the invention is used to determine when the user has achieved sleep or a beneficial level of rest. When the heart rate itself is lowered to a target resting heart rate level, the device starts a timed alarm to wake the user. Both the threshold target heart rate level and the duration of the sleep session may be determined by the user using input buttons to program the device.

The second application uses the heart rate to determine the duration of a sleep session. Users may use the device at night in this manner to measure the overall duration, and assess the quality, of their sleep. The measured data may be stored in the device's memory and accessed either by the user through the device or by the user's physician. The stored information may be related to the physician residing in a remote location. The results may be assessed for quality of sleep by recognizing when the heart rate is above or below the preset threshold target level as well as variations in the intervals between heart beats. Thus, the data may be used to determine whether or not the user is getting quality sleep, or is waking during sleep which is common in persons suffering from sleep apnea and heavy snoring. This information may be used by the user as a motivator to see a physician and/or a sleep specialist. This information is also valuable to the user's physician in determining if treatment is necessary and what type of treatment would be most effective. Subsequent impact of the treatment may also be evaluated using heart rate variability information.

The third application utilizes the heart rate to perform a heart rate variability test (HRV). HRV tests are typically performed while the subject is at rest or asleep or may be done over a user's normal 24-hour activities. User's can choose to have an HRV test performed using an input button. An HRV test may be performed in as little as ten seconds, but the longer the test, the more accurate the results. Users can utilize the HRV option while taking a timed nap, during a resting period, or when sleeping at night.

In the fourth basic application, the device is used in concert with a home's electronics control unit. Many homes are equipped with a controlling computer system. These homes have been referred to as ‘smart houses.’ The home's controlling computer or electronics control unit manages the functions of the home. These functions may include: television; personal computer; shower; home security system; lights; kitchen appliances; garage door and other functional features of a home. This invention is capable of working in concert with the home's controlling computer system and works to synchronize the home's functions with the homeowner's functions. The user wears the device before bed and when the user's heart rate level and variability reach the threshold level, the wrist worn monitor sends out a signal to the home's controlling computer which then prepares the home for the night, i.e., places the home in ‘sleep’ mode. This may comprise functions such as shutting lights and televisions off, ensuring the garage door is down, setting the thermostat at an appropriate temperature for the night, etc. The opposite is done in the morning. When the user's heart rate level and variability rises above the threshold level, the monitor sends a signal to the central home computer to prepare the home for the day, i.e., placing the home in ‘awake’ mode. Thus, functions such as turning on the lights, shower, coffee maker, alarm are accomplished. In addition to using the heart rate variability of the user to control the features

of the home, the monitor may have a button that manually accomplishes the tasks without use of heart rate variability information.

The present invention accomplishes these goals.

### SUMMARY OF THE INVENTION

A wrist-worn heart rate variability monitor is provided. Heart rate variability (“HRV”) refers to the interval between heart beats and is a reflection of an individual's current health status. Over time, an individual may use the results of HRV tests to monitor either improvement or deterioration of specific health issues. Thus, one use of the HRV test is as a medical motivator. When an individual has a poor HRV result, it is an indicator that they should consult their physician and make appropriate changes where applicable to improve their health. If an individual's HRV results deviate significantly from their normal HRV, they may be motivated to consult their physician. In addition, the inventive monitor is capable of monitoring the stages of sleep by changes in the heart rate variability and can record the sleep (or rest) sessions with the resulting data accessible by the user or other interested parties. The inventive monitor is thus capable of several novel uses: (1) to assist the user with a nap that allows predetermined time in one or more sleep stages; (2) determination of the duration of a sleep session, including length of time spent in one or more sleep stages; (3) in concert with a home's central electronic and computer control unit, the device uses HRV to determine when the house may be placed in “sleep” mode and when it is appropriate to place the house in “awake mode”; and (4) performance of an HRV test.

An object and advantage of the present invention is to provide a wrist worn heart rate variability monitor that is capable of timing sleep sessions and recording heart rate variability during the same.

Another object and advantage of the present invention is to provide a wrist worn heart rate variability monitor capable of performing a heart rate variability test.

Another object and advantage of the present invention is to provide a wrist worn heart rate variability monitor that allows the user to spend a predetermined amount of time in one or more sleep stages while recording the sleep session for future review and analysis.

Still another object and advantage of the present invention is to provide a wrist worn heart rate variability monitor that is capable of differentiating between the user's awake state, non-REM sleep state and REM sleep state.

Yet another object and advantage of the present invention is to provide a wrist worn heart rate variability monitor that allows recording of sleep sessions to determine and improve the quality and duration of the individual's sleep.

Another object and advantage of the present invention is to provide a wrist worn heart rate variability monitor that uses the obtained heart rate variability information to remotely instruct a central home computer to place the home in “sleep” mode when the monitor determines that the user falls asleep.

Another object and advantage of the present invention is to provide a wrist worn heart rate variability monitor that uses the obtained heart rate variability information to remotely instruct a central home computer to place the home in “awake” mode when the monitor determines that the user has awakened.

Another object and advantage of the present invention is to provide a wrist worn heart rate variability that is capable of detecting and recording sleep apnea events.

The foregoing objects and advantages of the invention will become apparent to those skilled in the art when the following detailed description of the invention is read in conjunction with the accompanying drawings and claims. Throughout the drawings, like numerals refer to similar or identical parts.

### BRIEF DESCRIPTION OF THE DRAWINGS

FIG. 1 is a top view of the wrist worn monitor.

FIG. 2 is a bottom view of the wrist worn monitor with electrodes and wires in phantom.

FIG. 3 is a side view of one embodiment of the wrist worn monitor closure.

FIG. 4 is a side view of the wrist worn monitor.

FIG. 5 is a bottom view of the wrist worn monitor illustrating possible two piece manufacture.

FIG. 6 is a top view of the membrane attachment.

FIG. 7 illustrates the membrane attached to the wrist worn monitor.

FIG. 8A is a bottom view illustrating placement of the alarm elements.

FIG. 8B is a top view illustrating placement of the alarm elements.

FIG. 9 is a view of the wrist worn monitor display.

FIG. 10 is a block diagram of the circuitry.

FIG. 11 is a block diagram of the communications unit with data transfer options.

FIG. 12 is a graphical representation of the heart rate.

FIG. 13 is a flowchart for using the wrist worn monitor to take a timed and recorded nap of specified duration.

FIG. 14 is a flowchart for using the wrist worn monitor to take a timed and recorded nap with a specified duration in REM sleep stage.

FIG. 15 is a flowchart for using the wrist worn monitor to take a timed and recorded nap with alarmed exit when REM sleep stage recognized.

FIG. 16 is a flowchart for using the wrist worn monitor to record Heart Rate Variability and time to analyze sleep duration and quality.

FIG. 17 is a flowchart for using the wrist worn monitor to monitor for and record Heart Rate Variability for sleep apnea events.

FIG. 18 is a flowchart for sending the heart rate variability data obtained by the wrist worn monitor to a central home computer to place the home in “sleep” and “awake” modes.

FIG. 19 is a flowchart for using the wrist worn monitor to perform a Heart Rate Variability (HRV) test.

### DETAILED DESCRIPTION OF THE INVENTION

The present invention is capable of monitoring, recording and analyzing sleep and/or rest sessions. The device monitors an individual's heart rate variability while the user is either at rest or asleep or physically active and records the results for up to 24 hours. The inventive monitor is capable of detecting and measuring the variability of heart rate during the sleep sessions and is further capable of discerning the subtle differences in heart rate variability as the user transitions from one sleep stage to the next. This record is stored in the device's memory and is accessible for review by the user or interested 3^{rd} parties such as the user's physician or nurse.

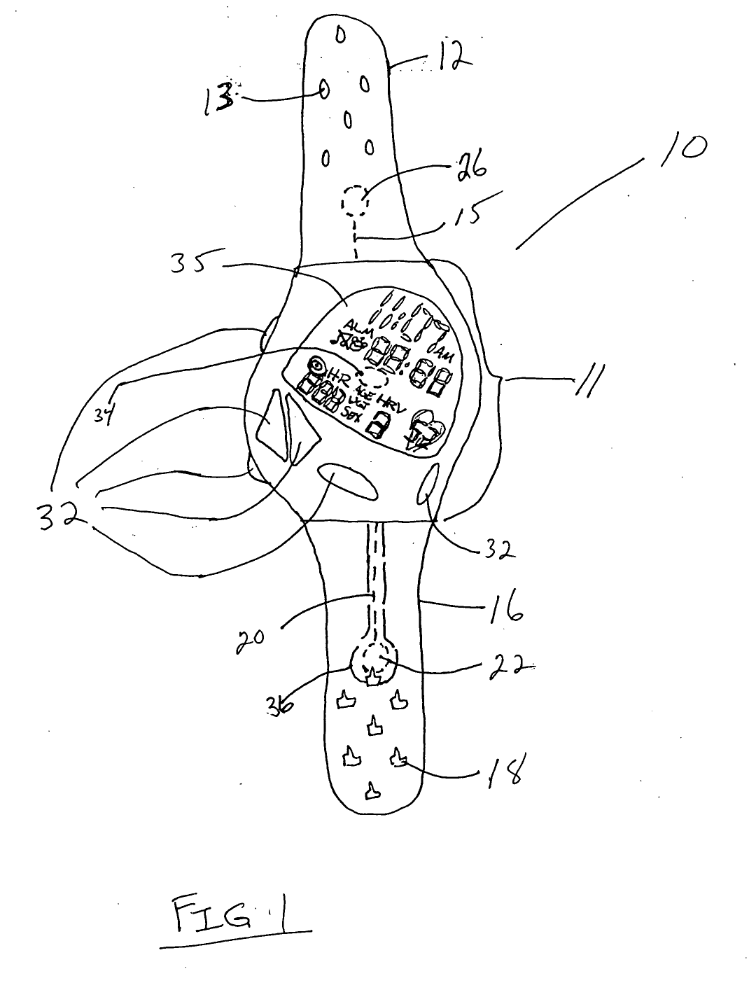

With reference to the accompanying Figures, there is provided a wrist worn heart rate variability monitor 10. As shown in FIG. 1, the monitor 10 is comprised of the monitor body 11, wristband B 12 and wristband A 16. The attributes of wristband B 12 will preferably be comprised of securing holes 13, a waking prompt 26 and a wire 15 connecting the waking prompt 26 to the monitor body 11. The attributes of wristband A 16 will preferably be comprised of securing hooks 18, at least one wire 20, electrode A 20 and may include a plastic insert on the back of wristband A. The monitor body 11 will preferably comprise the control unit 51, electrode B 34, display 35, a waking prompt 26, remote emitter 28, clock 30 and input buttons 32. The monitor may have six input buttons 32 which collectively make up the input, though one skilled in the art will recognize that more or fewer input buttons 32 may be used to accomplish the desired goals described herein.

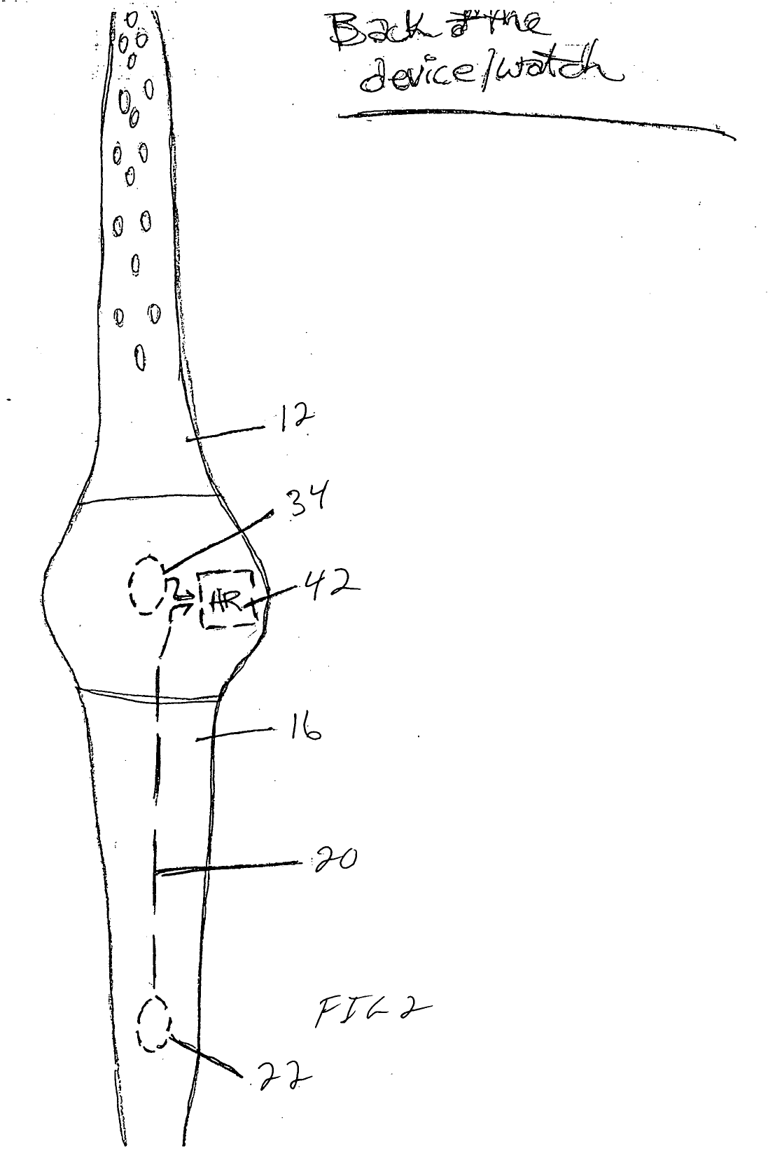

Turning now to FIG. 2, the inventive monitor 10 detects electrical signals generated by a body using at least two electrodes 22, 34, preferably the electrical signals are electrocardiograph (ECG) signals generated by the heart. Thus, in the preferred embodiment, the monitor 10 detects heart rate. This may be the type of heart rate monitor described in U.S. Pat. No. 5,738,104 or U.S. Pat. No. 5,876,350. The '350 patent discloses that the use of three electrodes is preferable to determine the heart rate to assist in filtering out undesirable noise attributable to the user's physiologic conditions while exercising, etc. Thus, if necessary, three electrodes may be used for the present invention, though the preferred embodiment utilizes two electrodes. Since the invention is designed for use while the user is either sleeping or at rest, the extraneous and undesirable noise associated with general physical activity by the user is not present, two electrodes is preferred. In an alternate embodiment not shown in the Figures, the heart beat signals may be detected using optical sensors.

The electrodes 22, 34 are integrated into the monitor. Electrode A 22 is housed in wristband A 16. Electrode A 22 may partially penetrate the surface of wristband A 16 or may be flush with the surface of wristband A 16. Electrode A 22 is connected with a wire(s) 200r fiber optic(s) thread(s) to the applicable unit for measuring the heart rate. These connective wire(s) 20 or thread(s) are housed in wristband A 16 and connect electrode A 22 to the monitor body 11 and in turn, to the applicable heart rate measuring device. Electrode B 34 is disposed on the back surface of the monitor body 12 so that it makes contact with the user's skin when worn. Electrode B 34 may protrude from the back surface of the monitor body 11 or, alternatively, it may be flush with the back surface of the monitor body 11.

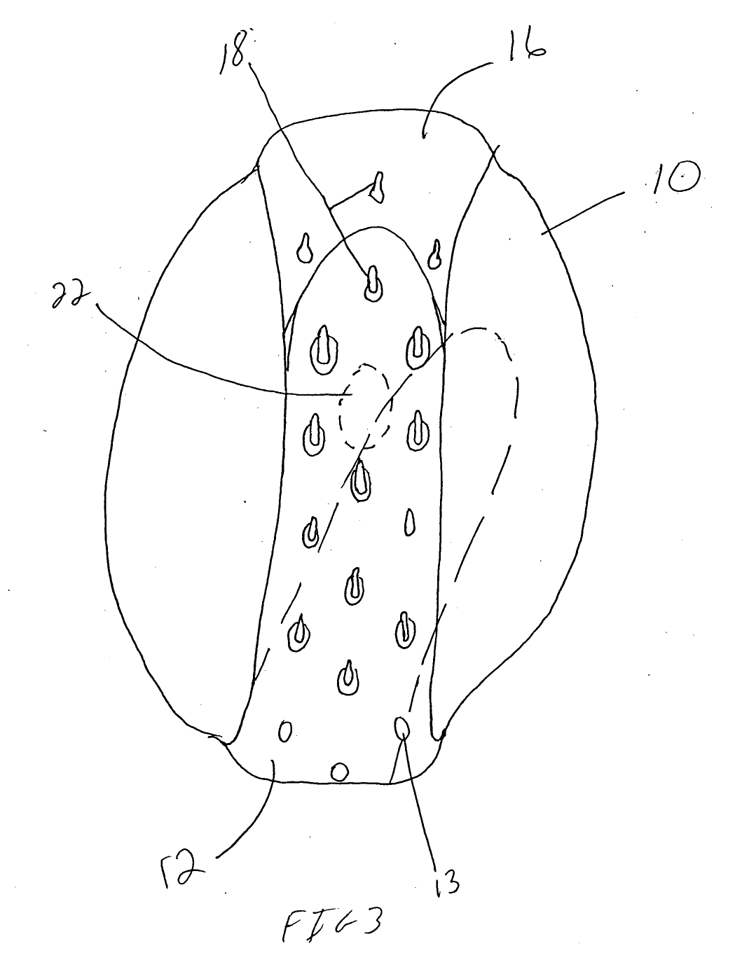

With reference to FIG. 3, the monitor 12 is attached to the user's wrist preferably using a system of holes 13 on wristband B 12 and securing hooks 18 on wristband A 16. The pliability of wristband B 12 allows the user to adjust the position of the securing points allowing electrode A 22 in wristband A 16 to have a proper fit and positioning for an accurate heart rate reading and further provides comfort on the user's wrist. Alternatively, the monitor 10 may be attached to the user's wrist by means of Velcro, buckle attachment, clasp, ball and hole, or other methods not shown in the Figures, but that are well known to those skilled in the art.

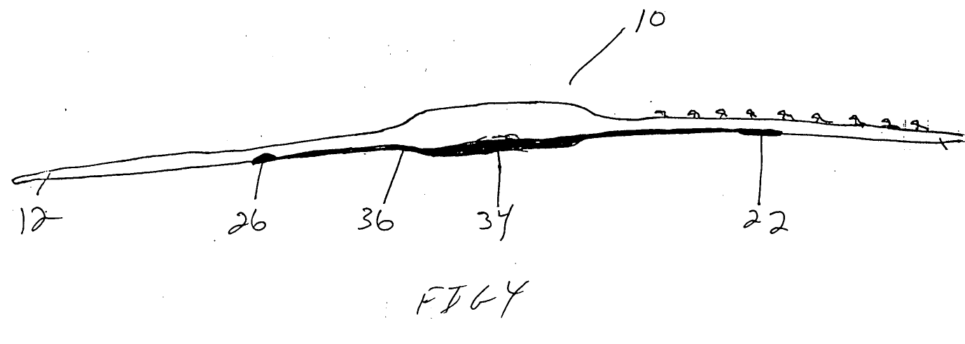

Turning now to FIG. 4, the monitor 10 may be largely constructed using technology that is conventional for construction of electronic watches. The monitor 10 will most likely be constructed of different types of plastic that range from rigid to pliable. Wristband B 12 may be made of different material than used in wristband A 16. The material in wristband B 14 may be more pliable than the material in wristband A 16 and vice versa. Such technology is not described herein in detail because it is well known to those skilled in the art.

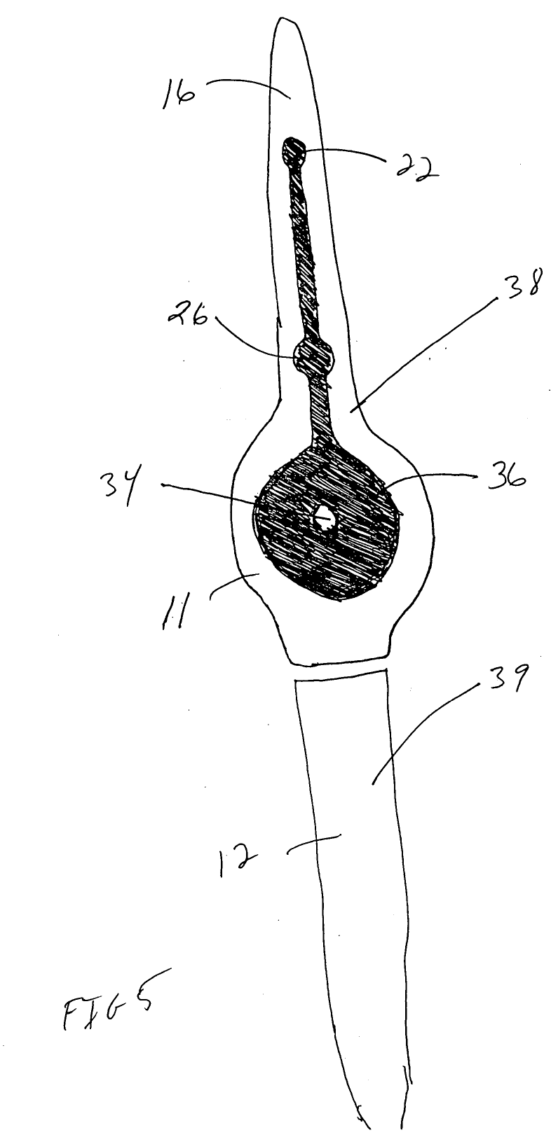

As indicated in FIG. 5, the monitor 10 may be made of two pieces. The monitor may be built using several different methods. It may have a pliable piece of plastic 36 that is inserted on the back side of the device sealing electrode A 22 into wristband A 16, electrode B 34 into the monitor body 11 and the waking prompt 26 into wristband B 12. One piece 38 may combine the monitor body 11 and wristband A 16. Wristband A 16 would house both the waking prompt 26 and electrode A 22. The second piece 39 would consist of a wristband B 12 and would be connected to the monitor body 11. The pliable plastic insert 36 may not need to cover electrode B 34. In both of these cases, the pliable plastic insert 36 would cover electrode A 22 and possibly electrode B 34 respective to the use of the insert 36. The connectivity method between wristband B 12 and the monitor body 11 is not discussed further as it is well known to those skilled in the art. Additionally, other common forms of manufacture are not described herein as they are well known to those skilled in the art.

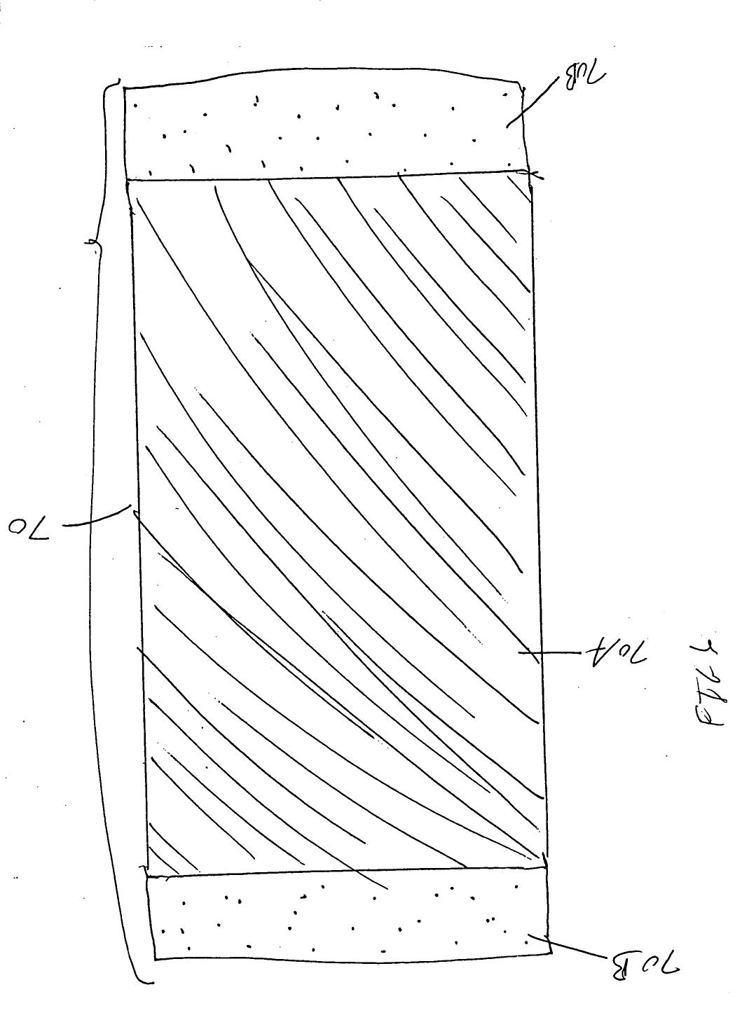

As illustrated in FIG. 6, a conductive membrane 40 may be attached to the back surface of the monitor 10 to increase the electrical conductivity, thus enhancing the monitor's ability to pick up the electrical signals generated by the heart. The membrane 40 may also be attached to the monitor's wristband covering the electrodes and having contact with the user's skin. The membrane 40 may be porous and may be used in concert with conductive gels. In this embodiment, the user will place a small amount of gel onto the membrane 40. The membrane will absorb the gel and the conductive properties of the gel will assist the electrodes 22, 34 in obtaining more accurate heart rate variability information. Preferably, the membrane 40 will retain the gel for multiple uses, thus eliminating the need for repeated applications of the gel to the membrane 40. The membrane 40 may also be constructed of conductive materials, thus eliminating the need for conductive gel. The membrane 40 will also benefit the fit of the electrode to the user's skin by eliminating or minimizing the space between the electrode and the user's skin.

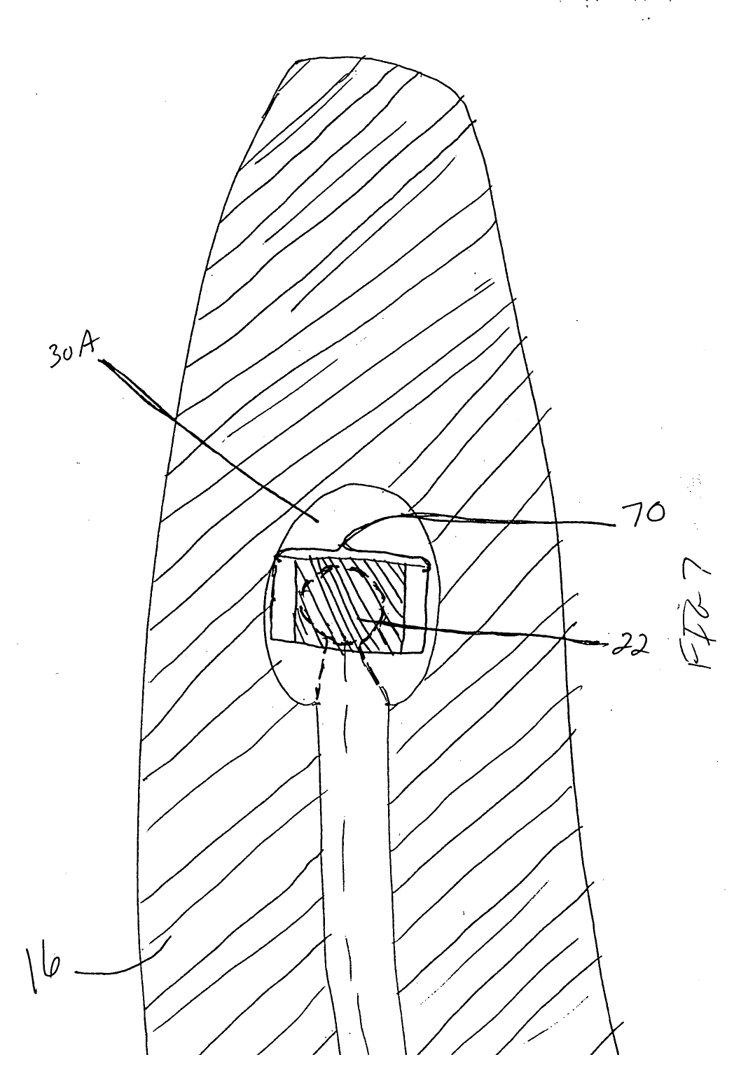

FIG. 7 illustrates the preferred embodiment for placement of the conductive membrane 40. The membrane 40 self-adheres to wristband A 16. A portion of wristband A 16 surrounding electrode A 22 will be smoothed out, thus ensuring good adhesion of the membrane 40. The membrane 40 is replaced when necessary by simply removing the used membrane 40 and applying a new membrane 40.

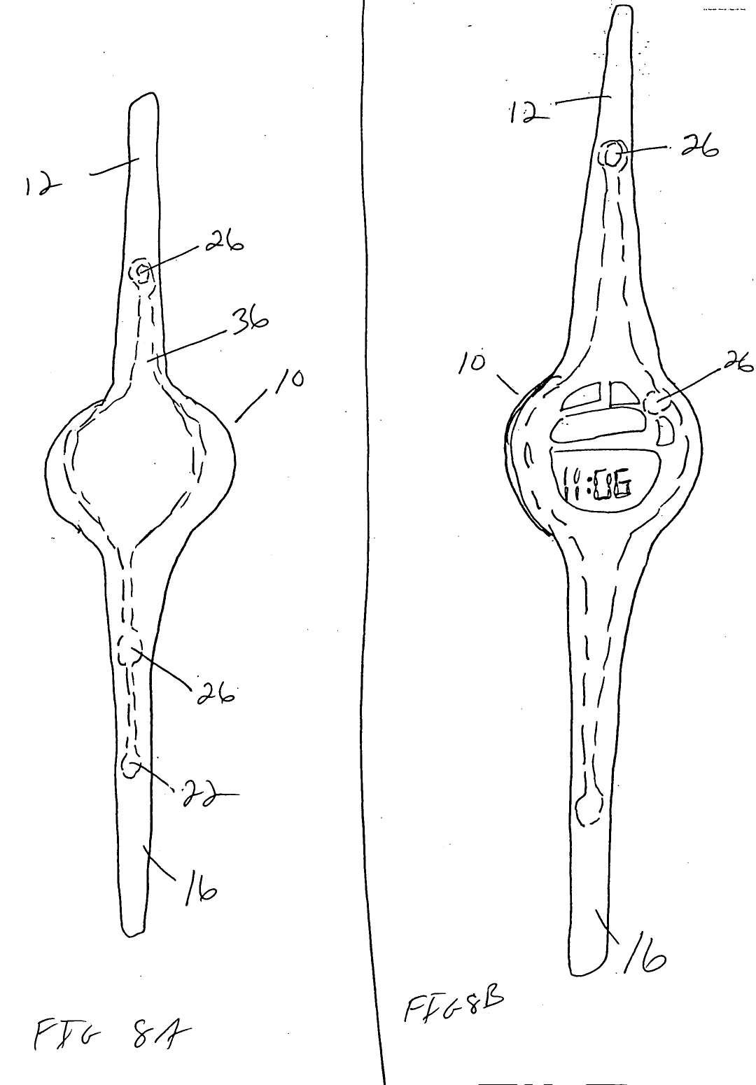

FIGS. 8A and 8B provide detail on the waking prompt 26 or alarm. The waking prompt 26 may be audible, silent through use of vibrations or emitted light. The vibrate alarm may be of the type described in either U.S. Pat. No. 4,456,387 or U.S. Pat. No. 5,400,301. The waking prompt 26 may also be partially housed in the pliable plastic insert 36 and housed in wristband B 12. Alternatively, the waking prompt 26 is housed in the monitor body 11. FIG. 8A illustrates housing the waking prompt in wristband A 16. Alternatively, the alarm unit may be housed in wristband A 16 using the pliable plastic insert 36. An audible or vibrational, or a combination thereof, alarm embodiment may be housed in the monitor body 11 or either wristband 12, 16 as discussed above.

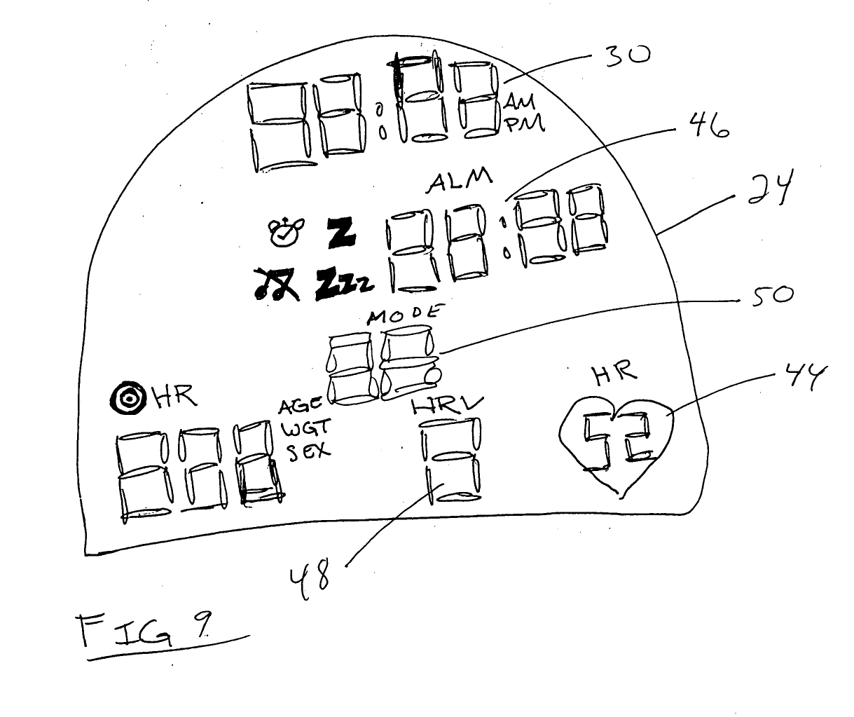

Turning now to FIG. 9, a particular embodiment of the display 35 is illustrated. The monitor 10 will preferably generate an optical gauge or display 35. The display 35 will preferably assist the user to set the monitor 10 to the desired modes and functions. The attributes of the display 35 may include a running real time clock 39 and allow the user to view their heart rate 44, alarm settings 46, heart rate variability test results 48, recorded rest time results, and the mode of the monitor 50.

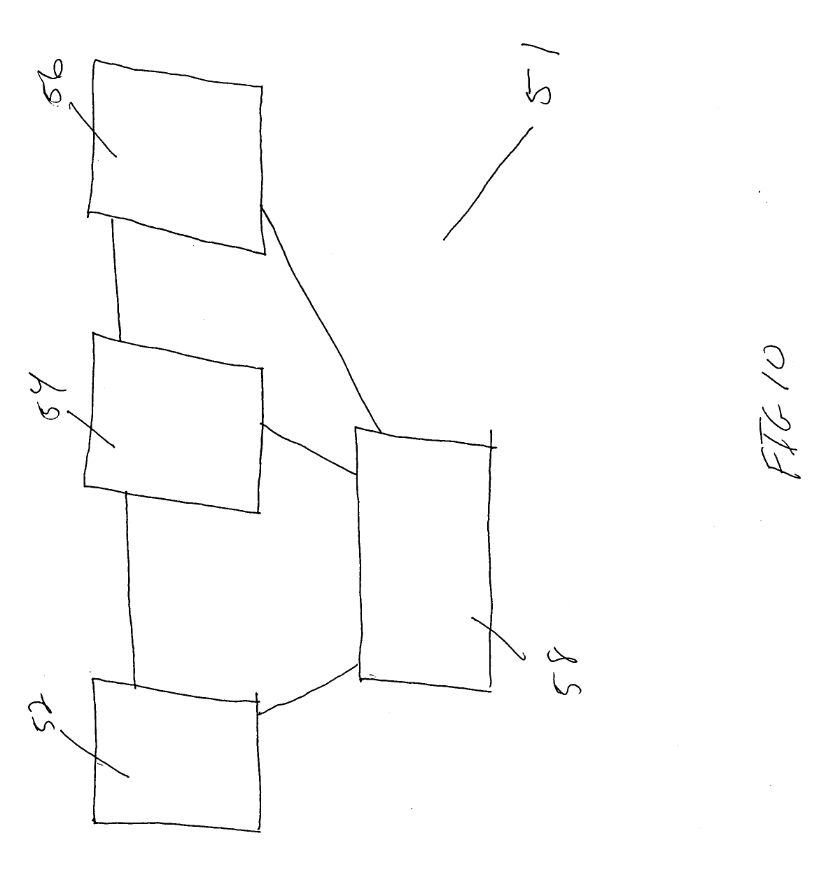

The exterior of the inventive monitor having been described, the internal circuitry will now be described. FIG. 10 provides a block diagram of the general circuitry blocks 51 and the interconnection thereof. The preferred embodiment thus provides an analog circuit block 52, a digital controller block 54, a communications block 56 and a power supply and power management block 58. Essentially, the electrodes pick up ECG (electrocardiograph) signals from the heart. The ECG signal is then conditioned to remove undesirable attributes, i.e., noise, from the signal. The analog signal is converted to a digital signal and then digitally processed under the software algorithms of the invention. The invention is capable of storing 24 hours of real time data. The details of the electronic circuitry are well known in the art and are not further described herein.

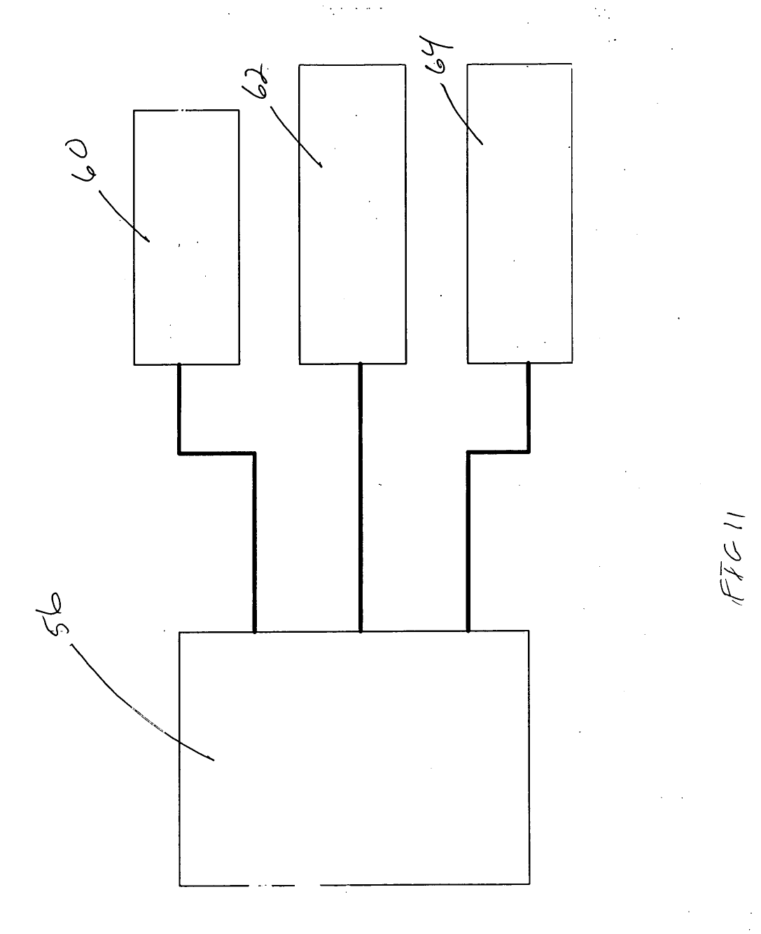

FIG. 11 is a block diagram of the communications block 56 interconnected with different external communication methods. It is desirable and useful to be able to either store the acquired data internally within the device, externally or to transmit it to external devices. Therefore, it is contemplated that conventional, preferably high speed, communications with external devices is an aspect of the present invention; it is contemplated that at least three types of transceivers accomplish this objective, each transceiver having different attributes and utility. For direct connection to a personal computer for further review, study and analysis of the data, high speed wired links are contemplated in the form of the direct connect USB 2.0 port 60. For ambulatory data transfer, wireless links are contemplated 62. For example, connection to a wireless communications devices, e.g., a Bluetooth® wireless device, may be provided. Alternatively, wireless USB 3.0 wireless ports are contemplated for uploading the acquired data. In addition, compatibility with certain medical instruments and notebook personal computers, an infrared transceiver 64 is provided as part of the watch design. The infrared method provides a slow, but proven and direct view optical link. Additional methods of transferring data from the inventive monitor will readily present themselves to those skilled in the art.

The hardware of the invention having been described, the operation of the invention will now be described.

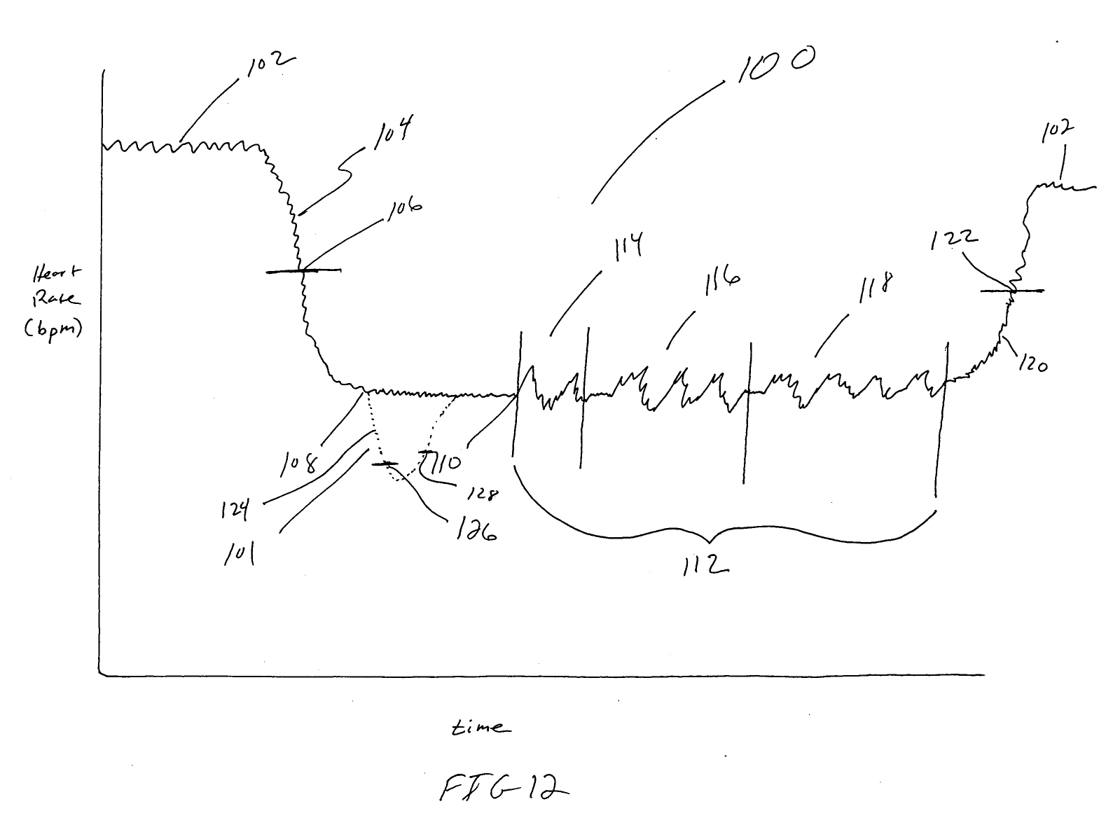

FIG. 12 illustrates typical heart rate variability 100 and includes typical heart rate data during a sleep apnea event in phantom 101. As discussed above, analysis of 24-hour HRV typically shows a nocturnal increase in the standard deviation of heart beat intervals. The heart rate and associated heart rate variability are essentially stable during the awake stage 102. The heart rate decreases significantly and rapidly 104 as the person begins to fall asleep. The heart rate eventually levels off, and the heart rate variability decreases, as a person eventually transitions 106 from the awake stage 102 to the non-REM stage 108. The heart rate variability remains relatively stable during the non-REM sleep stage 108.

As the individual eventually transitions from the non-REM sleep stage 108 to REM sleep 112, the heart rate becomes more erratic and the associated variability increases. There are several stages of REM sleep 112, each marked by changes in heart rate variability. FIG. 12 illustrates the first three REM stages, stage 1 114, stage 2 116, and stage 3 118. Typically, the first REM stage 114 lasts about 10 minutes, with each recurring REM stage 116, 118 lengthening, with the final stage lasting about one hour. The inventive monitor 10 is capable of detecting the heart rate variability within each sleep stage as well as the transition from one sleep stage to the next, i.e., the transition 106 from awake 102 to non-REM sleep 108, the transition 1010 from non-REM sleep 108 to REM sleep 112, and the completion of an REM sleep stage and subsequent transition to the next REM sleep stage.

Ultimately, the person exits REM sleep 112 and begins to awaken. This transition 122 is marked by an increase in heart rate 120 and is recognized by the monitor 10 when the heart rate increase passes a defined threshold 110, e.g., three standard deviations above the REM sleep state heart rate mean value. Eventually, the heart rate attains the stable awake stage 102 once more.

The heart rate data is processed in the digital processor component according to the computer program software code algorithms programmed therein. The essential theory of operation is that the heart rate data is first acquired by the monitor over a defined time interval. Typically at this stage, the user is in the awake state 102. The software then evaluates the heart rate itself and the variability of the interval between heart beats within a selected time period. Awake parameters are then calculated, comprising the mean awake heart rate value and standard deviation thereof. Alternatively, a heart rate threshold parameter may be entered by the user, corresponding to the user's resting heart rate, below which the user is recognized by the monitor as having fallen asleep. The user's heart rate, and associated variability, is next monitored and evaluated against the awake parameters, or the pre-entered threshold parameter, either periodically or continuously for significant changes. Specifically, the monitor is evaluating the user's heart rate for indication of the user's transition 106 from the awake state 102 to the non-REM sleep state 108. This transition 106 is marked by a decrease in heart rate 104 and is recognized by the device when the heart rate decrease passes a defined threshold 106, e.g., three standard deviations below the awake sleep state heart rate mean value. The threshold values of +/−three standard deviations from the local mean heart rate values are for illustrative purposes only. Those skilled in the art will readily comprehend that a number of threshold values may be used, depending on the particular user, etc.

As discussed above, the heart rate slows, and heart rate variability decreases when the user leaves the awake stage 102 and enters the non-REM sleep stage 108. Thus, when the awake-to-non-REM sleep threshold is reached 106, e.g., the user's heart rate drops below three standard deviations below the awake heart rate mean, the software recognizes this event as the user entering the non-REM sleep stage 108. Next, a new set of non-REM sleep parameters are calculated, including a mean non-REM heart rate and non-REM standard deviation over a defined time interval. The user's heart rate and associated variability is then monitored and evaluated against the non-REM sleep parameters, either periodically or continuously for significant changes.

The next event in the user's sleep cycle, assuming no interruptions in sleeping pattern, results in the user exiting non-REM sleep 108 and entering the first REM sleep stage or cycle 114. As described above, the transition from non-REM to REM sleep 110 results in an increase in the heart rate variability. Thus, when, e.g., the user's heart rate variability increases above a threshold level, e.g., the standard deviation about the mean increases by a factor of two as compared with the non-REM sleep standard deviation, the software recognizes this event as the user entering the REM sleep stage. Again, one skilled in the art will recognize that certain individuals may require a standard deviation factor increase that is either larger or smaller than a factor of two greater than the non-REM sleep standard deviation. A new set of REM sleep parameters are calculated, including an REM mean heart rate and an REM standard deviation over a defined time interval. The user's heart rate and associated variability is then monitored and evaluated against the REM sleep parameters, either periodically or continuously for significant changes.

Next, the user may exit REM sleep 112, in which case the heart rate increases significantly to cross a pre-defined threshold, e.g., more than three standard deviations over the mean REM sleep heart rate mean. The software is capable of recognizing on this basis that the user is now awake. The monitor is further capable of recognizing outlying data points resulting from transient events, e.g., the sleeping user physically changing positions, where the heart rate is temporarily increased, but rapidly returns to a level within the normal local deviation.

Alternatively, the user may exit the first REM sleep cycle 114, but instead of waking up will revert back to non-REM sleep 108 for a small amount of time and then enter the second, longer REM sleep cycle 116. The software is capable of recognizing the completion of one or more REM sleep cycles by differentially comparing the two sets of heart rate variability parameters. Ultimately, the user awakens and the heart rate increases such that the software recognizes the exit from REM sleep 112 and the awakened state. 122

Sleep apnea events may occur during either non-REM 108 or REM sleep 112 and are characterized by cessation of breathing and concomitant decrease in heart rate. FIG. 12 illustrates the decrease in heart rate during non-REM sleep in phantom 101. The monitor is capable of detecting these apnea events when a pre-defined threshold is crossed by the user's heart rate, e.g., the user's heart rate decreases more than two standard deviations from the relevant sleep stage mean heart rate value over a defined time interval 126. One skilled in the art will readily recognize that the most appropriate time interval is dependent upon a number of factors known in the art. The monitor is further capable of recording the apnea event data for subsequent review by the user and/or a physician. For example, the user may wake to find that six apnea events occurred during the sleep period and use this information as a motivation to see his or her physician. An alternate embodiment provides a waking prompt that activates to bring the user out of the apnea event. The waking prompt 26 may be audio, visual, or vibratory. A further alternate embodiment provides remote transmission of the waking prompt to a 3^{rd} person or remote device so that the 3^{rd} person is alerted to the user's apnea event(s).

With this basic algorithmic theory in place for the software, many inventive applications present themselves.

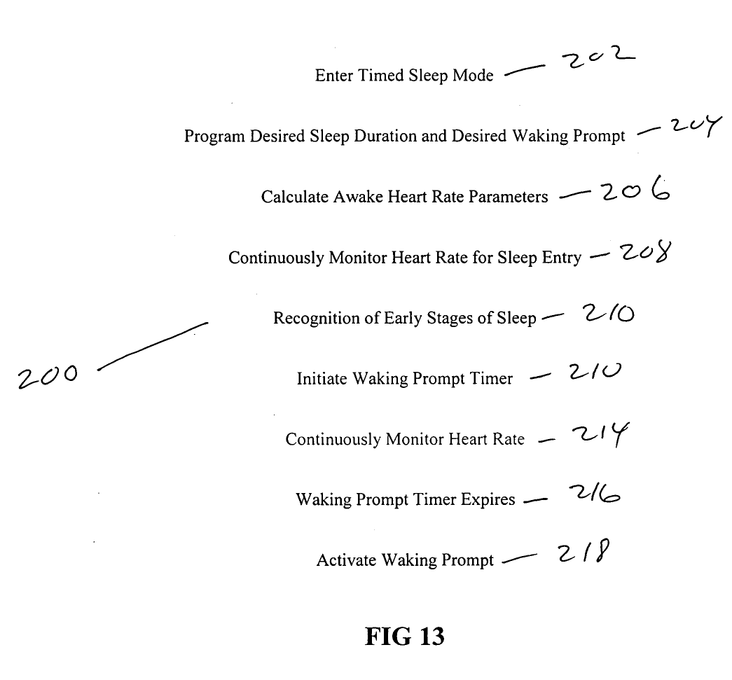

With specific reference to FIG. 13, the monitor is capable of allowing the user to take a nap of specified duration 200. The user selects timed-sleep mode 202 and enters the desired sleep duration and desired waking prompt 204. The waking prompt can be, as described above, either an audio, visual or vibrational alarm that is built into the monitor. The monitor acquires a signal of acceptable quality corresponding to the heart beat and begins to monitor for a particular time interval and ultimately calculates awake heart rate mean and standard deviation parameters 206. The preferred embodiment uses electrodes to acquire the ECG signals, however, an alternate embodiment may include the use of optical sensors to acquire the signal. The monitor then continuously, or periodically, monitors the heart rate for significant change, e.g., a 3 standard deviation decrease in heart rate from its local mean value, i.e., the awake mean in this case 208. When the monitor recognizes this change 210, it indicates that the user is now in the early stages of non-REM sleep and the waking prompt timer is started 212. The monitor then monitors and records the heart rate and associated variability 214 until either the user wakes and manually exits the selected mode or the waking prompt timer expires 216 which activates the waking prompt 218 and the heart rate monitoring is ended.

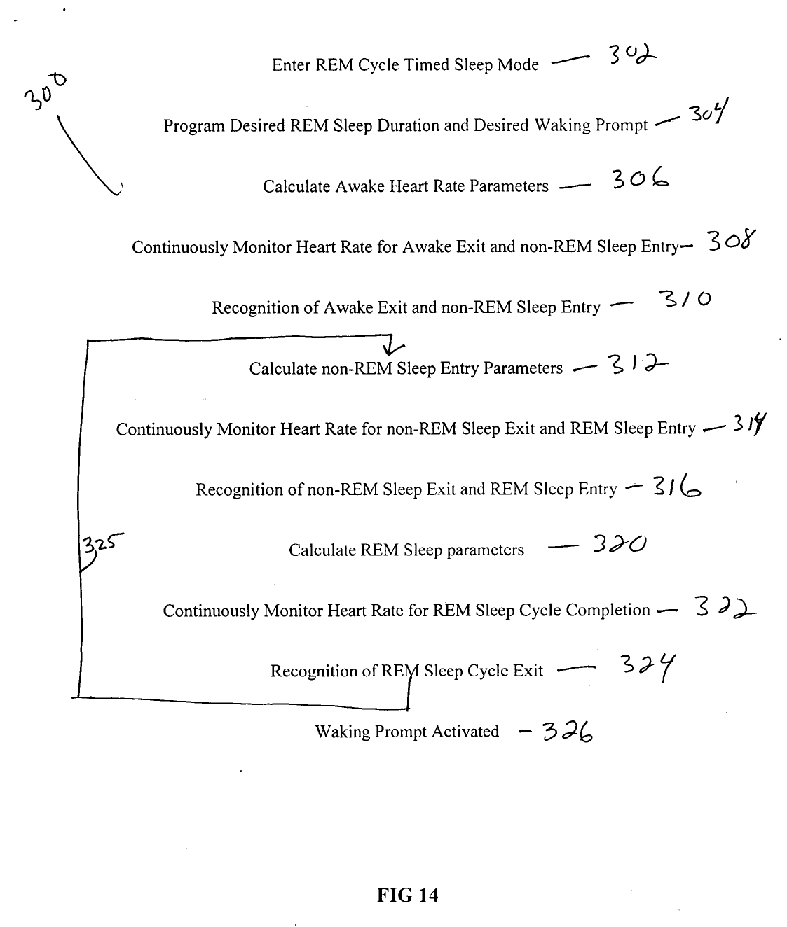

The next inventive method 300 is illustrated in FIG. 14. Here, the monitor also allows the user to exit a nap at a specified point. The difference is that the duration is not specified, rather the user specifies that they wish to be awoken after one or more REM sleep stages or cycles are completed. Thus, the user enters the REM cycle timed sleep mode 302, awake heart rate parameters are calculated 306 and heart rate monitored for sleep entry 308 as above. When non-REM sleep is recognized 310, non-REM sleep heart rate parameters calculated 312 and monitored for REM sleep entry 314 as described above. When REM sleep is recognized 316, REM sleep heart rate parameters are calculated 320 and monitored for completion of the desired numbers of REM sleep stages or cycles 322. One or more REM sleep cycles may be monitored and completed under this operational mode using a looping algorithm 325. When the desired numbers of REM sleep cycles are completed 324 the waking prompt is activated 326 to wake the user.

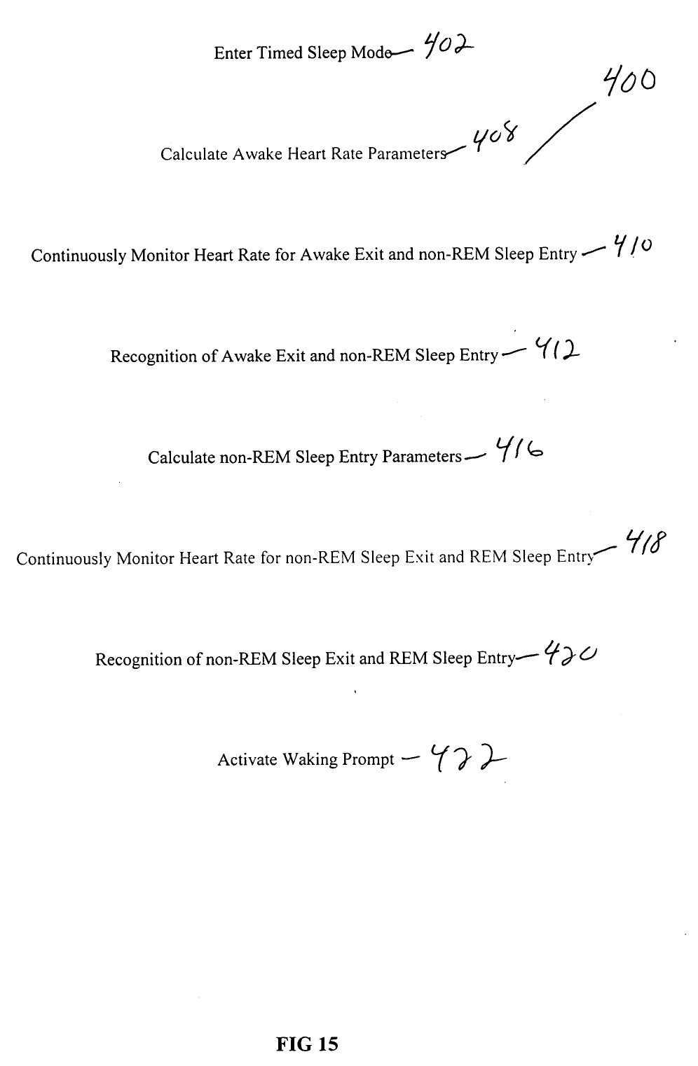

A further modification of the durationally limited nap is illustrated by FIG. 15. Here, the user desires to be awaked before falling deeply into the first REM sleep stage or cycle to avoid feeling groggy upon awakening 400. Thus, the user enters timed sleep mode 402, the awake heart rate parameters are calculated 408 and monitored for non-REM sleep entry 410 as above. When non-REM sleep is recognized 412, non-REM sleep parameters are calculated 416 and monitored for non-REM sleep exit 418 as described above. When the monitor recognizes that the user is exiting non-REM sleep 420 the waking prompt is activated 422 to wake the user.

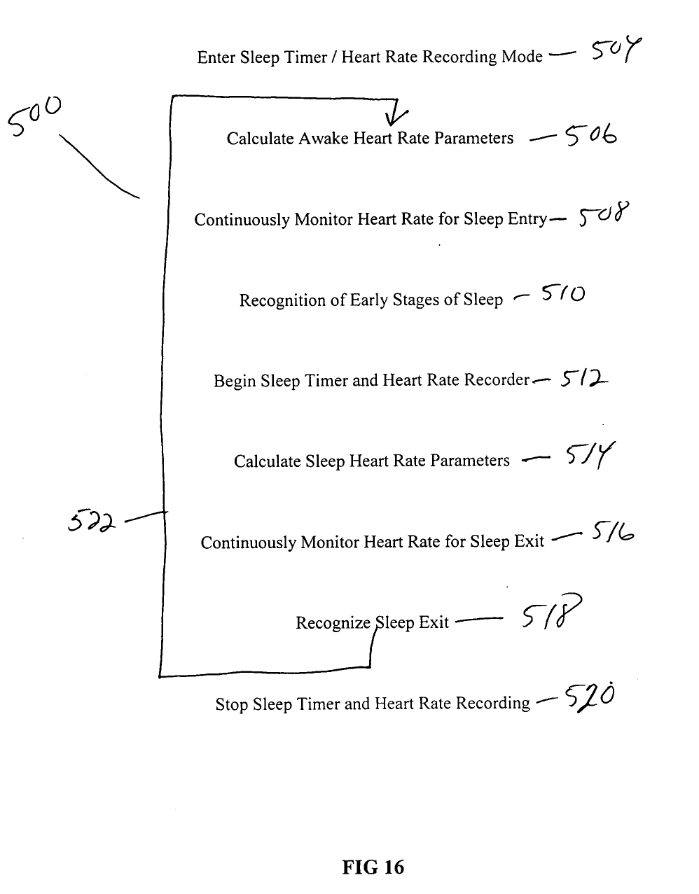

FIG. 16 provides a method of monitoring both the duration and quality of a user's normal sleeping routine 500. In this mode, the user enters the sleep timer/heart rate recording mode 504, the awake heart rate parameters are calculated 506 and monitored for non-REM sleep entry 508 as above. Upon recognition of non-REM sleep entry 510, the sleep timer and heart rate and variability recorder are activated 512. Sleep heart rate parameters are calculated 514 and monitored 516 for sleep exit. When sleep exit is recognized 518, i.e., the user awakens, the sleep timer and recording of heart rate are stopped 520. In an alternate embodiment, a loop in the algorithm 522 allows for repeating of the previous logic steps in case the user awakens in the middle of the night and then falls asleep once more. This general recording of heart rate and variability thereof allows the user and/or physician to view the time-stamped events of the night for sleep duration and quality, i.e., time spent in non-REM and/or the REM sleep stages or cycles with the ability to view sleep interruption events.

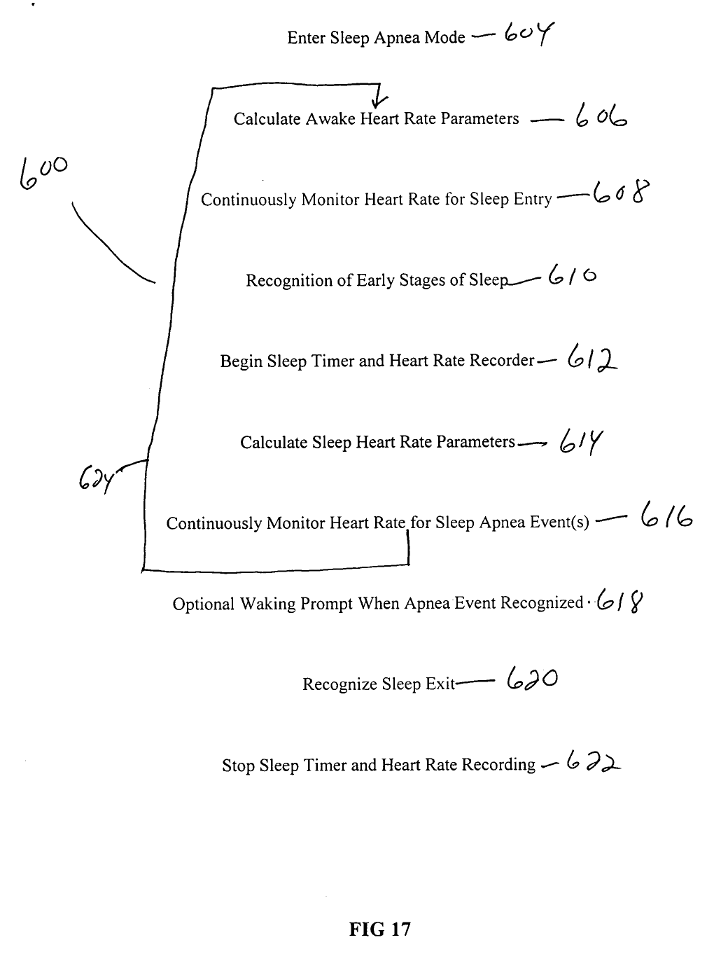

Turning now to FIG. 17, the monitor is used to detect sleep apnea events 600. In this case, the user enters sleep apnea monitoring mode 604, the awake heart rate parameters are calculated 606 and monitored 608 for sleep entry as above. Once sleep entry is recognized 610, the sleep timer and heart rate recorder are prompted to begin 612. Sleep heart rate parameters, including the stages for non-REM and REM sleep stages, are calculated 614 and monitored 616 as above. The monitor is, in this case, monitoring for deviations below the sleep heart rate parameters which are diagnostic of sleep apnea events 101 as indicated in FIG. 12. The intent of this inventive method is to record the apnea events for later review by the user and/or physician to assist in diagnosing sleep apnea and to assist in monitoring the effectiveness of treatment options. The monitor has the capability, in the preferred embodiment, to stop the sleep timer and heart rate recording 622 when sleep exit is recognized 620 and, as above, restart the timer and recording if the user falls back asleep as illustrated by the looping algorithm 624. This capability is particularly important if the apnea event causes the user to come out of the sleep state. As discussed above, alternate embodiments include a waking prompt 618, either audio, visual or vibratory, that will wake the user upon detection of an apnea event. Alternatively, an alarm signal may be transmitted to a 3^{rd} person alerting them of the user's apnea event(s). Finally, the number of apnea events may be displayed for the user, thus providing motivation to see their physician.

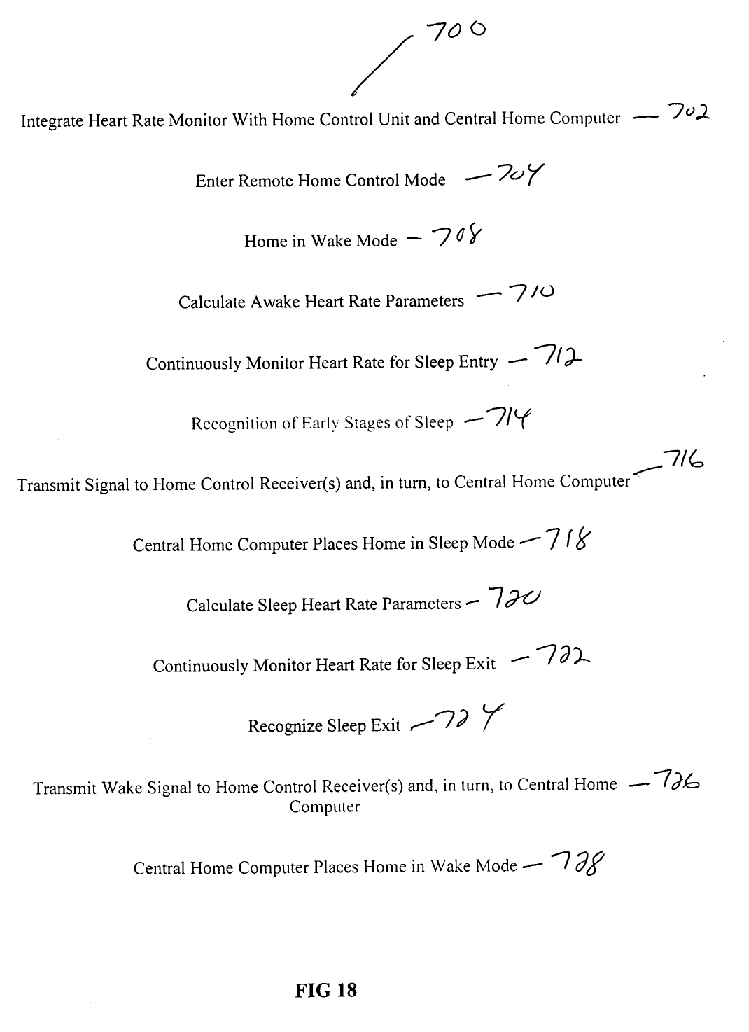

FIG. 18 illustrates one embodiment of the monitor's ability to assist in controlling a home's functional features based on heart rate variability 700. In this embodiment, the monitor is used in concert with a home's electronics control unit 702. Many homes are equipped with a controlling computer system. These homes have been referred to as ‘smart houses.’ The home's controlling computer or electronics control unit manages the functions of the home. These functions may include: television; personal computer; shower; home security system; lights; kitchen appliances; garage door and other functional features of a home. This invention is capable of working in concert with the home's controlling computer system and works to synchronize the home's functions with the homeowner's functions. The user enters remote home control mode 704 and, with the home in ‘wake’ mode 708, wears the device before bed. The awake parameters are calculated 710 and monitored 712 as above. When sleep is recognized as discussed above 714, the wrist worn monitor sends out a signal to the home's controlling computer via a home control receiver(s) 716, which then prepares the home for the night, i.e., places the home in ‘sleep’ mode 718. This may comprise functions such as shutting lights and televisions off, ensuring the garage door is down, setting the thermostat at an appropriate temperature for the night, etc. The opposite is done in the morning. Thus, the sleeping user's heart rate parameters are calculated as above 720 and monitored 722 for sleep exit 724. When the user's heart rate level and variability rises above the threshold level, i.e., sleep exit is recognized 724, the monitor sends a signal to the central home computer via the home control receiver(s) 726 to prepare the home for the day, i.e., placing the home in ‘awake’ mode 728. Thus, functions such as turning on the lights, shower, coffee maker, alarm are accomplished. In addition to using the heart rate variability of the user to control the features of the home, the monitor may have a button that manually accomplishes the tasks without use of heart rate variability information.

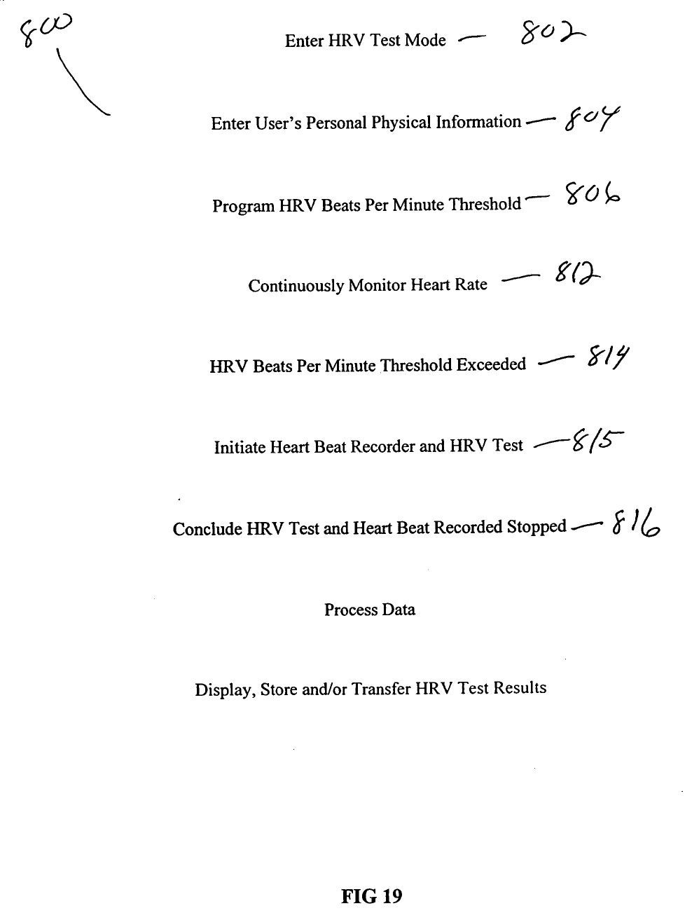

FIG. 19 provides another application of the invention. A heart rate variability test may be taken by the monitor 800. Here, the user enters the HRV testing mode 802 and then enters personal physical information 804 which may affect the test results such as age, sex, weight. A target heart rate threshold is entered by the user and desired duration of the test 806. The target heart rate threshold may be either an upper or lower threshold. The test may be administered either while the user is at rest, while the user sleeps, either in non-REM sleep stage only or in REM sleep stage only or across both sleep stages, or during physical activity. The monitor then monitors the heart rate 812 until the target lower threshold is crossed which either indicates that the user has attained a resting level or, alternatively, has entered the non-REM sleep stage, or, if the monitor is used in connection with physical activity, an upper target heart rate threshold is utilized. In either case, the monitor initiates the heart beat recorder and the HRV test commences 815 for a specified time once the target heart rate threshold is crossed 814. The longer the HRV test, the more accurate the results will be. When the specified duration is reached, the HRV test concludes 816 and the monitor then processes the data 818. The data is preferably displayed on a scale of 1-200 to indicate the quality of the user's HRV 820. Alternatively, a scale from 1-10 may be used or letters, e.g., A, B, C, etc., or even colors like green (good HRV), yellow (marginal HRV), red (poor HRV) may be used.

The monitor further provides the capability, through use of selective input of operational modes, performance of one or more of the above-described functions in parallel, at the same time, during a single monitoring session.

The above specification describes certain preferred embodiments of this invention. This specification is in no way intended to limit the scope of the claims. Other modifications, alterations, or substitutions may now suggest themselves to those skilled in the art, all of which are within the spirit and scope of the present invention. It is therefore intended that the present invention be limited only by the scope of the attached claims below:

1. A wrist worn heart rate variability monitor, comprising:
at least two electrical contacts for detecting analog electrical signals generated within a body when placed in contact with the body;
a circuit that conditions the electrical signals and converts the analog electrical signals to digital signal data;
a heart rate variability signal processor that monitors and analyzes the digital signal data and obtains heart rate variability data therefrom; and
a memory capable of storing at least 24 hours of real time digital signal data.

2. The apparatus of claim 1, further comprising the electrical signals being ECG signals from the heart.

3. The apparatus of claim 1, further comprising a processor that is capable of performing a heart rate variability test.

4. The apparatus of claim 1, further comprising a processor that is capable of performing a heart rate variability test while a user sleeps.

5. The apparatus of claim 1, further comprising a processor that is capable of performing a heart rate variability test while a user is awake and resting.

6. The apparatus of claim 1, further comprising a processor that is capable of performing a heart rate variability test while a user is physically active.

7. The apparatus of claim 1, further comprising a processor that is capable of analyzing the heart rate variability to determine when the user is asleep and then performs a heart rate variability test during the sleep period.

8. The apparatus of claim 1, further comprising a timer, wherein the timer is capable of timing the duration of the monitoring of the heart rate variability data and time-stamping the data.

9. The apparatus of claim 3, further comprising a timer, wherein the timer is capable of timing the duration of the heart rate variability test.

10. The apparatus of claim 8, further comprising a waking prompt capable of activation when a specified time for monitoring the heart rate variability has passed, and wherein the processor stops monitoring heart rate variability when the waking prompt is activated.

11. The apparatus of claim 1, wherein the processor differentiates between a user's awake and sleep stages based upon heart rate variability data.

12. The apparatus of claim 11, wherein the processor recognizes differentiation between a user's awake state and non-REM sleep state based upon heart rate variability data.

13. The apparatus of claim 12, further comprising a waking prompt, wherein the waking prompt is activated when non-REM sleep state is recognized.

14. The apparatus of claim 11 wherein the processor recognizes differentiation between a non-REM sleep state and a REM sleep state based upon heart rate variability data.

15. The apparatus of claim 14, further comprising a waking prompt, wherein the waking prompt is activated when REM sleep state is recognized.

16. The apparatus of claim 14, further comprising a processor that is capable of performing a heart rate variability test during the non-REM sleep state and stopping the test when the REM sleep state is recognized.

17. The apparatus of claim 14, further comprising a processor that is capable of discerning and counting REM sleep state cycles and wherein the waking prompt is activated after a specified number of REM sleep state cycles are completed by a user.

18. The apparatus of claim 1, further comprising a processor capable of monitoring heart rate variability data during a user's sleep period and wherein a sleep apnea event may be detected therefrom.

19. The apparatus of claim 1, further comprising a waking prompt, wherein the waking prompt is activated when a sleep apnea event is detected.

20. The apparatus of claim 2, further comprising the monitor having a back surface; and a conductive membrane disposed on the back surface of the monitor and having contact with the user's skin to increase the monitor's ability to pick up the ECG signals.

21. The apparatus of claim 20, further comprising the conductive membrane being porous.

22. The apparatus of claim 21, further comprising conductive gel, the conductive gel being incorporated into the pores of the conductive membrane to increase the monitor's ability to pick up the ECG signals.

23. The apparatus of claim 1, for the control of appliances installed in each room, comprising:
home information transmission paths from the wrist worn heart rate monitor to each room;
at least one home control unit receiver, connectable to the transmission paths, installed in selected rooms for transmitting and receiving information along the transmission paths, the wrist worn heart rate variability monitor capable of transmitting an awake signal or a sleep signal to the at least one home control unit receiver based upon heart rate variability data;
a central home control unit, connectable to the transmission paths, the at least one home control unit receiver and to appliances in the rooms, whereby the control unit receives the awake or sleep signal transmitted by the at least one control unit receiver, wherein when an awake signal is transmitted to the appliances by the computer, the appliances are turned on and when a sleep signal is transmitted by the computer, the appliances are turned off.

24. The apparatus of claim 23, further comprising the home information transmission pathways capable of receiving wireless transmission of the from the monitor, the pathways wirelessly transmitting the wake or sleep signal to the central home control unit and the pathways wirelessly transmitting the wake or sleep signal to the home appliances.

25. The apparatus of claim 23, further comprising the home information transmission pathways capable of receiving electronic transmission of wake or sleep signal from the from the monitor, the pathways electronically transmitting the wake or sleep signal to the central home control unit and the pathways electronically transmitting the wake or sleep signal to the home appliances.

26. A Wrist worn heart rate variability monitor, comprising:
at least two electrical contacts for detecting ECG signals generated by a body's heart when placed in contact with the body;
a circuit that conditions the electrical signals and converts the analog signal to a digital signal;
a memory capable of storing 24 hours of real time digital signal data;
a heart rate variable signal processor that monitors and analyzes the digital data and obtains heart rate variability data therefrom;
the processor further capable of performing a heart rate variability test, the processor further capable of differentiating between a user's awake and sleep stages based upon heart rate variability data;
a timer, the timer capable of timing the duration of the monitoring of the heart rate variability data; and
a waking prompt, the waking prompt capable of activation when REM sleep is recognized.

27. A Wrist worn heart rate variability monitor, comprising:
optical sensors for detecting ECG signals generated by a body's heart when placed in contact with the body;
a circuit that conditions the electrical signals and converts the analog signal to a digital signal;
a memory capable of storing 24 hours of real time digital signal data;
a heart rate variable signal processor that monitors and analyzes the digital data and obtains heart rate variability data therefrom;
the processor further capable of performing a heart rate variability test, the processor further capable of detecting a sleep apnea event based upon heart rate variability data;
a timer, the timer capable of timing the duration of the monitoring of the heart rate variability data; and
a waking prompt, the waking prompt capable of activation when a sleep apnea event is recognized.

28. A method for monitoring heart rate variability using a wrist worn heart rate variability monitor, comprising:
detecting electrical signals generated from a body by the body's heart;
analyzing the signals to determine heart rate variability; and
monitoring and storing the heart rate variability data.

29. The method of claim 28, further comprising performing a heart rate variability test.

30. The method of claim 28, further comprising:
analyzing the heart rate variability data to determine when the user is asleep; and
performing a heart rate variability test while the user is asleep.

31. The method of claim 28, further comprising performing a heart rate variability test while the user is awake and resting.

32. The method of claim 28, further comprising timing the monitoring of the heart rate variability data.

33. The method of claim 28, further comprising differentiating between an awake state and non-REM and REM sleep stages using heart rate variability data.

34. The method of claim 33, further comprising timing the duration of the sleep stages.

35. The method of claim 34, further comprising time-stamping the heart rate variability data.

36. The method of claim 33, further comprising waking the user after recognition of entry into REM sleep state.

37. The method of claim 33 further comprising:
recognizing the completion of at least one REM sleep state cycle; and
waking the user after recognizing the completion of one or more REM sleep state cycles.

38. The method of claim 33, further comprising:
recognizing non-REM sleep;
transmitting a signal from the monitor to at least one home control unit receiver;
transmitting a signal from the at least one home control unit receiver to a central home computer;
placing home in sleep mode based on instructions from the central home computer;
monitoring for sleep exit;
recognizing sleep exit;
transmitting a signal from the monitor to the at least one home control unit receiver;
transmitting a signal from the at least one home control receiver to the central home computer; and
placing home in awake mode based on instructions from the central home computer.

39. A method for monitoring heart rate variability using a wrist worn heart rate variability monitor, comprising:
detecting electrical signals from a body by the body's heart;
analyzing the signals to determine heart rate variability;
monitoring and storing the heart rate variability data;
analyzing the heart rate variability data to determine when the user is asleep;
differentiating between an awake state, non-REM sleep state and REM sleep state; and
waking the user after recognition of entry into the REM sleep state.

40. The method of claim 28, further comprising:
detecting a sleep apnea event.

41. The method of claim 40, further comprising:
transmitting an alarm to a 3^{rd} party, alerting them of the sleep apnea event.

42. A computer program product for monitoring heart rate variability using a wrist worn heart rate variability monitor, comprising:
detecting electrical signals generated from a body by the body's heart;
analyzing the signals to determine heart rate variability; and
monitoring, analyzing and storing the heart rate variability data.

43. The computer program product of claim 42, further comprising performing a heart rate variability test.

44. The computer program product of claim 42, further comprising analyzing the heart rate variability data to determine when the user is asleep; and
performing a heart rate variability test while the user is asleep.

45. The computer program product of claim 42, further comprising performing a heart rate variability test while the user is awake and resting.

46. The computer program product of claim 42, further comprising timing the monitoring of the heart rate variability data.

47. The computer program product of claim 44, further comprising timing of the duration of the performance of the HRV test.

48. The computer program product of claim 42, further comprising differentiating between an awake state and non-REM and REM sleep stages using heart rate variability data.

49. The computer program product of claim 48, further comprising waking the user after recognition of entry into REM sleep state.

50. The computer program product of claim 48, further comprising:
recognizing the completion of at least one REM sleep state cycle; and
waking the user after the recognizing the completion of at least one REM sleep state cycle.

51. The computer program product of claim 48, further comprising:
recognizing non-REM sleep;
transmitting a signal from the monitor to at least one home control unit receiver;
transmitting a signal from the at least one home control unit receiver to a central home computer;
placing home in sleep mode based on instructions from the central home computer;
monitoring for sleep exit;
recognizing sleep exit;
transmitting a signal from the monitor to the at least one home control unit receiver;
transmitting a signal from the at least one home control receiver to the central home computer; and
placing home in awake mode based on instructions from the central home computer.

52. The computer program product of claim 42, further comprising recognizing a sleep apnea event.
